(19) United States
(12) Patent Application Publication
(10) Pub. No.: US 2021/0059964 A1
(43) Pub. Date: Mar. 4, 2021

(54) Anti-I-Type Allergy Agent, Degranulation Inhibitor for Basophils and Mast Cells, Anti-Dementia Agent, Agent for Improving/Inhibiting Short-Term Memory Impairment

(71) Applicant: Mitsui Sugar Co., Ltd., Tokyo (JP)

(72) Inventors: Toma Furuta, Chuo-ku, Tokyo (JP); Masami Mizu, Chuo-ku, Tokyo (JP)

(73) Assignee: Mitsui Sugar Co., Ltd., Tokyo (JP)

(21) Appl. No.: 16/962,255

(22) PCT Filed: Jan. 23, 2019
(86) PCT No.: PCT/JP2019/002110

§ 371 Date: Jul. 15, 2020

(30) Foreign Application Priority Data
Jan. 24, 2018 (JP) 2018-009872
Mar. 14, 2018 (JP) 2018-046752

(51) Int. Cl. A61K 31/192; A61P 25/28

(52) U.S. Cl. CPC A61K 31/192; A61K 9/0053; A61P 25/28

(57) ABSTRACT

According to an aspect of the present invention, there is provided an anti-I-type allergy agent including: p-coumaric acid as an active ingredient. In addition, according to another aspect of the present invention, there is provided an anti-dementia agent including: p-coumaric acid as an active ingredient.

### TECHNICAL FIELD

The present invention relates to an anti-I-type allergy agent, a degranulation inhibitor for basophils and mast cells, an anti-dementia agent, and an agent for improving/inhibiting short-term memory impairment.

### BACKGROUND ART

Allergies are defined as “systemic or local damage to living bodies based on immune reactions.” Allergy reactions are classified into types I to IV. Of these, type I to III allergies are related to humoral immunity involving serum alternation, and a type IV allergy is related to cellular immunity due to sensitized lymphocytes.

A type I allergy is also called an immediate-type allergy or anaphylaxis-type allergy. Examples of symptoms of type I allergies include hay fever and urticaria. Since there is a relatively large population having symptoms of these, an effective anti-I-type allergy agent is required. For example, Patent Literature 1 discloses a cosmetic material or an external skin preparation which has an antiallergic effect using a carnitine derivative and/or the carnitine derivative in combination with an effect promoter.

On the other hand, dementia refers to a state in which various disorders occur due to death of cells in the brain or deterioration of functions of the brain due to various causes, thereby interfering with life. In some cases, atrophy of the entire brain accompanied by the onset of dementia may lead to loss of physical functions.

In recent years, research has been actively performed on components having an effect of inhibiting dementia. For example, Patent Literature 2 discloses that royal jelly exhibits anti-dementia activity.

### CITATION LIST

### Patent Literature

[Patent Literature 1] Japanese Unexamined Patent Publication No. 2014-114289
[Patent Literature 2] PCT International Publication No. WO2017/078175

### Non Patent Literature

[Non Patent Literature 1] Folia Pharmacologica Japonica, 130, (2), pp. 112 to 116, 2007

### SUMMARY OF INVENTION

### Technical Problem

An object of the present invention is to provide a novel anti-I-type allergy agent and a degranulation inhibitor for basophils and mast cells. In addition, another object of the present invention is to provide a novel anti-dementia agent and an agent for improving/inhibiting short-term memory impairment.

### Solution to Problem

According to an aspect of the present invention, there is provided an anti-I-type allergy agent including: p-coumaric acid as an active ingredient.

p-Coumaric acid is one of hydroxycinnamic acids classified as a polyphenol and is known to have effects such as an antioxidant action. The present inventors conducted in vitro tests and have found that a composition containing p-coumaric acid has an anti-I-type allergic action. That is, according to the anti-I-type allergy agent of the present invention, symptoms of type I allergies can be inhibited (treated, alleviated, or prevented).

The anti-I-type allergy agent of the present invention may be based on a degranulation inhibitory action of basophils and mast cells.

The anti-I-type allergy agent of the present invention has at least an action of inhibiting degranulation of basophils and mast cells. Accordingly, according to the anti-I-type allergy agent of the present invention, symptoms of type I allergies can be effectively inhibited.

According to another aspect of the present invention, a degranulation inhibitor for basophils and mast cells which includes p-coumaric acid as an active ingredient is provided.

In addition, the present inventors have conducted in vivo tests and have found that p-coumaric acid has an action of improving/inhibiting short-term memory impairment caused by accumulation of amyloid β proteins.

That is, according to still another aspect of the present invention, there is provided an anti-dementia agent including: p-coumaric acid as an active ingredient.

The present invention can also be regarded as providing an agent for improving/inhibiting short-term memory impairment including: p-coumaric acid as an active ingredient.

### Advantageous Effects of Invention

According to the present invention, it is possible to provide a novel anti-I-type allergy agent and a degranulation inhibitor for basophils and mast cells. In addition, according to the present invention, it is possible to provide a novel anti-dementia agent and an agent for improving/inhibiting short-term memory impairment.

### BRIEF DESCRIPTION OF DRAWINGS

FIG. 1 is a graph showing degranulation rates of Examples 1 to 3, Comparative Example 1, and a positive control.

FIG. 2 is a graph showing evaluation results in Y-shaped maze tests.

### DESCRIPTION OF EMBODIMENTS

Hereinafter, embodiments of the present invention will be described. However, the present invention is not limited to the following embodiments.

An anti-I-type allergy agent according to an embodiment and an anti-dementia agent according to an embodiment contain p-coumaric acid as an active ingredient.

p-Coumaric acid is a kind of hydroxycinnamic acid and is available as a lignin degradation product, a product separated from natural essential oils, a synthetic reaction product, or the like. Lignin derived from gramineous plants or lignin derived from sugar cane or bagasse may be used as lignin which is a raw material of a lignin degradation product. A commercially available p-coumaric acid may be used.

<Anti-I-Type Allergy Agent>

An anti-I-type allergy agent of the present specification is a composition having an action of inhibiting type I allergy symptoms. The action of inhibiting type I allergy symptoms may be, for example, an action of alleviating, treating, or preventing symptoms, such as hay fever, urticaria, allergic rhinitis, and bronchial asthma, due to type I allergies. In addition, the action of inhibiting type I allergy symptoms may be an action of inhibiting degranulation of basophils and mast cells in a mechanism of a type I allergic reaction to be described below. That is, the anti-I-type allergy agent of the present specification may be a degranulation inhibitor for basophils and mast cells.

The mechanism of the type I allergic reaction is as follows.

(1) In a case where an antigen (allergen) such as pollen or a mite enters a living body, helper T cells (Th2 cells) give an instruction to differentiate B-cells into immunoglobulin E (IgE) antibody-producing cells.

(2) IgE antibodies specific to antigens of IgE antibody-producing cells are produced from the IgE antibody-producing cells.

(3) In a case where IgE antibodies bind to mast cells or basophils and antigens bind thereto again, chemical mediators such as histamine and leukotriene are secreted (degranulated), whereby allergic symptoms develop.

The anti-I-type allergy agent of the present embodiment particularly has an action (degranulation inhibitory action) of inhibiting extracellular release (degranulation) of granules containing chemical mediators such as histamine and leukotriene from mast cells or basophils in a type I allergic reaction. For this reason, according to the anti-I-type allergy agent of the present embodiment, symptoms due to a type I allergic reaction can be effectively inhibited, treated, or prevented.

Whether or not an anti-I-type allergy agent has a degranulation inhibitory action can be confirmed through the following method. For example, cells, such as rat basophilic leukemia cells (RBL-2H3 cells), which extracellularly release granulocytes containing histamine through cross-linking of IgE bound on a cell surface due to an antigen are used. The extent to which the degranulation of a specimen to which an anti-I-type allergy agent is added is inhibited when these cells are stimulated by the antigen is calculated compared to a specimen to which no anti-I-type allergy agent is added.

The anti-I-type allergy agent according to the present embodiment may consist of only p-coumaric acid which is an active ingredient, or may further contain materials that can be used in foods, quasi-drugs, or pharmaceutical products. Materials that can be used in foods, quasi-drugs, or pharmaceutical products are not particularly limited, but examples thereof include amino acids, proteins, carbohydrates, oils and fats, sweeteners, minerals, vitamins, flavorings, excipients, binders, lubricants, disintegrators, emulsifiers, surfactants, bases, solubilizers and suspending agents.

Examples of proteins include milk casein, whey, soy protein, wheat protein, and egg white. Examples of carbohydrates include corn starch, cellulose, pregelatinized starch, wheat starch, rice starch, and potato starch. Examples of oils and fats include salad oil, corn oil, soybean oil, safflower oil, olive oil, and palm oil. Examples of sweeteners include sugars such as glucose, sucrose, fructose, glucose-fructose liquid sugar, and fructose-glucose liquid sugar, sugar alcohols such as xylitol, erythritol, and maltitol, artificial sweeteners such as sucralose, aspartame, saccharin, and acesulfame K, and natural sweeteners such as stevia. Examples of minerals include calcium, potassium, phosphorus, sodium, manganese, iron, zinc, and magnesium, and salts thereof. Examples of vitamins include vitamin E, vitamin C, vitamin A, vitamin D, and vitamin Bs, biotin, and niacin. Examples of excipients include dextrin, starch, lactose, and crystalline cellulose. Examples of binders include polyvinyl alcohol, gelatin, hydroxypropyl methyl cellulose, hydroxypropyl cellulose, sodium carboxymethyl cellulose, and polyvinyl pyrrolidone. Examples of lubricants include magnesium stearate, calcium phosphate, and talc. Examples of disintegrators include crystalline cellulose, agar, gelatin, calcium carbonate, sodium hydrogen carbonate, and dextrin. Examples of emulsifiers or surfactants include sucrose fatty acid esters, citrates, stearoyl lactate, glycerin fatty acid esters, polyglycerin fatty acid esters, sorbitan fatty acid esters, propylene glycol fatty acid esters, and lecithin. Examples of bases include cetostearyl alcohol, lanolin, and polyethylene glycol. Examples of solubilizers include polyethylene glycol, propylene glycol, sodium carbonate, and sodium citrate. Examples of suspending agents include glycerin monostearate, polyvinyl alcohol, polyvinyl pyrrolidone, methyl cellulose, hydroxymethyl cellulose, and sodium alginate. These may be used singly or in combination of two or more thereof.

In a case where an anti-I-type allergy agent is obtained by being formulated with another material, the content of p-coumaric acid which is an active ingredient may be appropriately set depending on the form or purpose of use of an anti-I-type allergy agent to be described below, but is preferably greater than or equal to 50 μg/g, more preferably greater than or equal to 70 μg/g, still more preferably greater than or equal to 100 μg/g, and preferably less than or equal to 1,000 μg/g, more preferably 800 μg/g, and still more preferably less than or equal to 700 μg/g based on the total amount of anti-I-type allergy agent from the viewpoint of more easily inhibiting degranulation of basophils and adipocytes.

The anti-I-type allergy agent can be used in foods, quasi-drugs, or pharmaceutical products. Foods may be provided in forms of, for example, health foods, specific health foods, functional foods, nutritionally functional foods, and supplements.

The anti-I-type allergy agent may be in any form of solids (such as powder or granules), liquids (such as solutions or suspensions), and paste, and may be in any dosage form such as powder agents, pills, granules, tablets, capsules, troches, liquid agents, and suspensions.

The anti-I-type allergy agent may be administered parenterally, but is preferably administered orally. Regarding a dose of the anti-I-type allergy agent, the anti-I-type allergy agent is preferably administered such that there is greater than or equal to 20 μg of p-coumaric acid per administration, more preferably administered such that there is greater than or equal to 30 μg of p-coumaric acid per administration, and still more preferably administered such that there is greater than or equal to 40 μg of p-coumaric acid per administration, for example. In addition, the anti-I-type allergy agent is preferably administered such that there is greater than or equal to 60 μg of p-coumaric acid per day, more preferably administered such that there is greater than or equal to 90 μg of p-coumaric acid per day, and still more preferably administered such that there is greater than or equal to 120 μg of p-coumaric acid per day. In addition, the anti-I-type allergy agent is preferably administered such that there is less than or equal to 150 mg of p-coumaric acid per administration, more preferably administered such that there is less than or equal to 100 mg of p-coumaric acid per administration, and still more preferably administered such that there is less than or equal to 80 mg of p-coumaric acid per administration. In addition, the anti-I-type allergy agent is preferably administered such that there is less than or equal to 450 mg of p-coumaric acid per day, more preferably administered such that there is less than or equal to 300 mg of p-coumaric acid per day, and still more preferably administered such that there is less than or equal to 200 mg of p-coumaric acid per day.

The anti-I-type allergy agent of the present embodiment has the above-described action, and therefore, can be used for patients with symptoms of type I allergies or those who have not yet developed allergies and desire to prevent type I allergies.

A specific aspect of a degranulation inhibitor for basophils and mast cells according to an embodiment may be the same as the aspect of the above-described anti-I-type allergy agent. That is, for the degranulation inhibitor for basophils and mast cells according to the embodiment, the “anti-I-type allergy agent” in the description relating to the above-described anti-I-type allergy agent may be replaced with the “degranulation inhibitor for basophils and mast cells.”

An embodiment of the present invention can be regarded as a method for inhibiting type I allergies or a method for inhibiting degranulation of basophils and mast cells which includes a step of administering an effective amount of the above-described anti-I-type allergy agent or degranulation inhibitor for basophils and mast cells, which contains p-coumaric acid as an active ingredient, to a subject in need thereof. In addition, an embodiment of the present invention can be regarded as p-coumaric acid for use in the method for inhibiting type I allergies or the method for inhibiting degranulation of basophils and mast cells. The subject in the above-described methods may be a mammal, and is preferably a human. The aspect, administration method, dose, and the like of the anti-I-type allergy agent or the degranulation inhibitor for basophils and mast cells may be the same as those described above.

An embodiment of the present invention can be regarded as use of p-coumaric acid for producing an anti-I-type allergy agent or producing a degranulation inhibitor for basophils and mast cells. In addition, an embodiment of the present invention can be regarded as use of p-coumaric acid for inhibiting type I allergies or inhibiting degranulation of basophils and mast cells. The aspect of the anti-I-type allergy agent or the degranulation inhibitor for basophils and mast cells may be the same as that described above.

<Anti-Dementia Agent>

The anti-dementia agent of the present invention has an anti-dementia action. The “anti-dementia action” of the present invention is a concept including an action of preventing the onset of dementia, an action of delaying the onset of dementia, and an action of allowing recovery from dementia once developed from the onset state. The dementia to be treated by the anti-dementia agent of the present invention may be Alzheimer's dementia. In Alzheimer's dementia, there is a pathological condition (choline hypothesis) caused by falling off of acetylcholinergic nerve cells in the Meynert nucleus of the basal forebrain, and a pathological condition (amyloid hypothesis) caused by accumulation of amyloid β proteins. The two pathological conditions are different from each other, and are not the same pathological condition seen from different angles. The dementia to be treated by the anti-dementia agent of the present invention may be Alzheimer's dementia based on any hypothesis, and is preferably Alzheimer's dementia based on the amyloid hypothesis. The dementia to be treated by the anti-dementia agent of the present invention may be Alzheimer's dementia caused by accumulation of amyloid β proteins. In other words, it can also be said that the present invention provides an anti-dementia agent for Alzheimer's dementia, an anti-dementia agent for Alzheimer's dementia based on the amyloid hypothesis, or an anti-dementia agent for Alzheimer's dementia caused by accumulation of amyloid β proteins.

Since death of cerebral nerve cells is caused by accumulation of tau proteins in addition to amyloid β proteins in the brain, it is thought that this causes impairment in cognitive function in Alzheimer's dementia based on the amyloid hypothesis. Accumulation of amyloid β proteins starts in an initial stage, and accumulation of tau proteins starts after about 10 years. Thereafter, amyloid β proteins and tau proteins continue to accumulate, thereby killing cerebral nerve cells, and it is thought that dementia develops about 25 years after the initial stage. According to another aspect, it can also be said that the anti-dementia agent of the present invention has an action of inhibiting accumulation of amyloid β proteins in the brain and an action of reducing the amount of amyloid β proteins accumulated in the brain, and it can also be said that the anti-dementia agent of the present invention has an action of inhibiting accumulation of tau proteins in the brain and an action of reducing the amount of tau proteins accumulated in the brain.

It can also be said that the present invention provides an agent for improving/inhibiting memory impairment. The agent for improving/inhibiting memory impairment of the present invention has an action of improving and/or inhibiting memory impairment. The “improving/inhibiting memory impairment” of the present invention is a concept including an action of preventing the onset of memory impairment, an action of delaying the onset of memory impairment, and an action of allowing recovery from memory impairment once developed from the onset state. The memory impairment to be treated by the agent for improving/inhibiting memory impairment of the present invention may be long-term memory impairment or short-term memory impairment, but is preferably short-term memory impairment. That is, it can be said that the present invention provides an agent for improving/inhibiting short-term memory impairment, and it can also be said that the present invention provides an agent for improving/inhibiting short-term memory impairment caused by accumulation of amyloid β proteins.

The anti-dementia agent according to the present embodiment may consist of only p-coumaric acid which is an active ingredient, or materials that can be used in foods, quasi-drugs, or pharmaceutical products may also be incorporated. As the materials that can be used in foods, quasi-drugs, or pharmaceutical products, the same materials that can be used in the above-described anti-I-type allergy agent may be used.

The anti-dementia agent can be used in foods, quasi-drugs, or pharmaceutical products. Foods may be provided in forms of, for example, health foods, specific health foods, functional foods, nutritionally functional foods, and supplements.

The anti-dementia agent can also be used in feed or a feed additive. Examples of feed include feed for companion animals such as dog food or cat food, livestock feed, poultry feed, and feed for cultured fish and shellfish. All feed ingredients orally taken by animals for nutritional purposes are included in “feed.” More specifically, coarse feed, concentrate feed, inorganic feed, and specialty feed are all included therein when feed is classified in terms of nutrient content, and compound feed, mixed feed, and single substance feed are all included therein when feed is classified in terms of official standards. In addition, feed for direct feeding, feed which is mixed with other feed, and feed which is added to drinking water for supplying nutrients are all included therein when feed is classified in terms of feeding methods.

The anti-dementia agent may be in any form of solids (such as powder or granules), liquids (such as solutions or suspensions), and paste, and may be in any dosage form such as powder agents, pills, granules, tablets, capsules, troches, liquid agents, and suspensions.

In a case where the above-described product containing an anti-dementia agent is intensively consumed, regarding the intake (intake or dose per day) of the above-described product, the above-described product is preferably consumed such that there is greater than or equal to 50 mg of p-coumaric acid per kg (body weight) and more preferably consumed such that there is greater than or equal to 100 mg of p-coumaric acid per kg (body weight), and is preferably consumed such that there is less than or equal to 3,000 mg of p-coumaric acid per kg (body weight) and more preferably consumed such that there is less than or equal to 2,000 mg of p-coumaric acid per kg (body weight). That is, in the case where the above-described product containing an anti-dementia agent is intensively consumed, regarding the intake (intake or dose per day) of the above-described product, the above-described product is preferably consumed such that there is 50 to 3,000 mg of p-coumaric acid per kg (body weight), 100 to 3,000 mg of p-coumaric acid per kg (body weight), 100 to 3,000 mg of p-coumaric acid per kg (body weight), or 100 to 2,000 mg of p-coumaric acid per kg (body weight).

In a case where the anti-dementia agent is taken on a daily basis for a long period of time, as the intake (intake or dose per day) of the above-described product, the above-described product is preferably consumed so that p-coumaric acid becomes greater than or equal to 1 mg/kg (body weight) and preferably consumed so that p-coumaric acid becomes less than or equal to 500 mg/kg (body weight).

The anti-dementia agent of the present embodiment can be used for humans or animals with an accumulation of amyloid β proteins. In addition, the anti-dementia agent of the present embodiment can be used for humans or animals suffering from dementia (or Alzheimer's dementia) or humans or animals suffering from memory impairment (or short-term memory impairment), and the dementia and memory impairment may be caused by accumulation of amyloid β proteins. The anti-dementia agent of the present embodiment can be used for preventing the onset of dementia or memory impairment in humans or animals that have not developed dementia (or Alzheimer's dementia) or in humans or animals that have not developed memory impairment (or short-term memory impairment), and can be used for delaying the onset of dementia or memory impairment.

A specific aspect of an agent for improving/inhibiting short-term memory impairment according to an embodiment may be the same as the aspect of the above-described anti-dementia agent. That is, for the agent for improving/inhibiting short-term memory impairment according to the embodiment, the “anti-dementia agent” in the description relating to the above-described anti-dementia agent may be replaced with the “agent for improving/inhibiting short-term memory impairment.”

An embodiment of the present invention can be regarded as a method for improving/inhibiting dementia or short-term memory impairment which includes a step of administering an effective amount of the above-described anti-dementia agent or agent for improving/inhibiting short-term memory impairment which contains p-coumaric acid as an active ingredient, to a subject in need thereof. In addition, an embodiment of the present invention can be regarded as p-coumaric acid for use in the method for improving/inhibiting dementia or short-term memory impairment. The subject in the above-described methods may be a mammal, and is preferably a human. The aspect, administration method, dose (intake), and the like of the anti-dementia agent or the agent for improving/inhibiting short-term memory impairment may be the same as those described above.

Furthermore, an embodiment of the present invention can be regarded as use of p-coumaric acid for producing an anti-dementia agent or an agent for improving/inhibiting short-term memory impairment. In addition, an embodiment of the present invention can be regarded as use of p-coumaric acid for improving/inhibiting dementia or short-term memory impairment. The aspect of the anti-dementia agent or the agent for improving/inhibiting short-term memory impairment may be the same as that described above.

### EXAMPLES

Hereinafter, the present invention will be described with reference to examples. However, the present invention is not limited by these examples.

<Test 1: Evaluation as Anti-I-Type Allergy Agent>

[Preparation of Test Solution] p-Coumaric acid was dissolved in ethanol to prepare 50 mg/mL test stock solutions. These test stock solutions were diluted with each buffer solution shown in the following Table 1, and test solutions having specimen concentrations of 1,000, 500, and 250 μg/mL were prepared.

[RBL-2H3 Cell Degranulation Inhibition Test]

(Test Operation)

RBL-2H3 cells, which were rat basophilic leukemia cells, (National Institutes of Biomedical Innovation, Health and Nutrition) were seeded in a 96-well plate and were then cultured overnight. Media which had a composition shown in Table 1 and contained an anti-DNP-IgE antibody were respectively added thereto, and each mixture was allowed to react at 37° C. for 2 hours. Then, the cells were washed with the buffer solutions. Furthermore, the prepared 1,000, 500, and 250 μg/mL test solutions were added thereto so that the final concentrations became 500 μg/mL (Example 1), 250 μg/mL (Example 2), and 125 μg/mL (Example 3), respectively. Thereafter, the mixtures were allowed to react at 37° C. for 10 minutes. Then, DNP-labeled human serum albumin was added thereto, and the mixtures were further allowed to react at 37° C. for 3 hours. In addition, as an untreated control (Comparative Example 1), the same test was carried out with only a buffer solution being added but no test solution being added, and, as a positive control, with Wortmannin (Wako Pure Chemical Industries, Ltd.) being added so that the final concentration became 25 nmol/L. In addition, an antigen-unstimulated control to which a medium containing no anti-DNP-IgE antibody was added and then a buffer solution and DNP-labeled human serum albumin were sequentially added to allow reaction in the same manner was used.

After fractionating the total amount of cell supernatant into empty wells, a lysis buffer was added to the cells, and the mixture was allowed to stand for 10 minutes at room temperature to obtain a cell lysate. A p-nitrophenyl-2-acetamido-2-deoxy-β-D-glucopyranoside solution (hereinafter, referred to as a substrate solution) was added to each of the cell supernatant and the cell lysate, and the mixtures were allowed to react at 37° C. for 25 minutes. Then, glycine buffer was added thereto to stop the reaction. In addition, sample blanks obtained by adding a glycine buffer to each of a cell supernatant and a cell lysate, allowing the mixtures to react at 37° C. for 25 minutes, and subsequently adding a substrate solution thereto were used.

TABLE 1

| Material | Composition |
|---|---|
| Medium | DMEM medium |
| | Fetal bovine serum (10 vol %, on the basis of |
| | total amount of medium) |
| | Penicillin-streptomycin solution (1 vol %, on the |
| | basis of total amount of medium) |
| Buffer solution (MT | NaCl (137 mmol/L) |
| buffer, pH 7.3) | KCl (2.7 mmol/L) |
| | CaCl₂ (1.8 mmol/L) |
| | MgCl₂•6H₂O (1 mmol/L) |
| | D(+)-glucose (5.6 mmol/L) |
| | HEPES Free acid (20 mmol/L) |
| | Albumin bovine F-V (1 g/L) |

[Calculation of Degranulation Rate]

For each sample of Examples 1 to 3, Comparative Example 1, a positive control, an antigen-unstimulated control, and sample blanks, the absorbance of p-nitrophenol generated by reaction between a substrate and β-hexosaminidase present in granules was measured (at a measurement wavelength of 405 nm and a control wavelength of 650 nm) using a microplate reader (SpectraMax M2e, Molecular Devices, LLC.)

The emission rate was obtained using the following equation from the absorbance of each sample with respect to the absorbance of Comparative Example 1, and each degranulation rate was calculated from the emission rate. In the equation, “absorbance on cell supernatant” and “absorbance on cell solution side” are values obtained by subtracting the sample blanks.

Emission rate (%)=absorbance on cell supernatant side/(absorbance on cell supernatant side+absorbance on cell solution side)

Degranulation rate (%)={average value of (emission rate of test solution−emission rate of antigen-unstimulated control)/(emission rate of untreated control−emission rate of antigen-unstimulated control)}×100

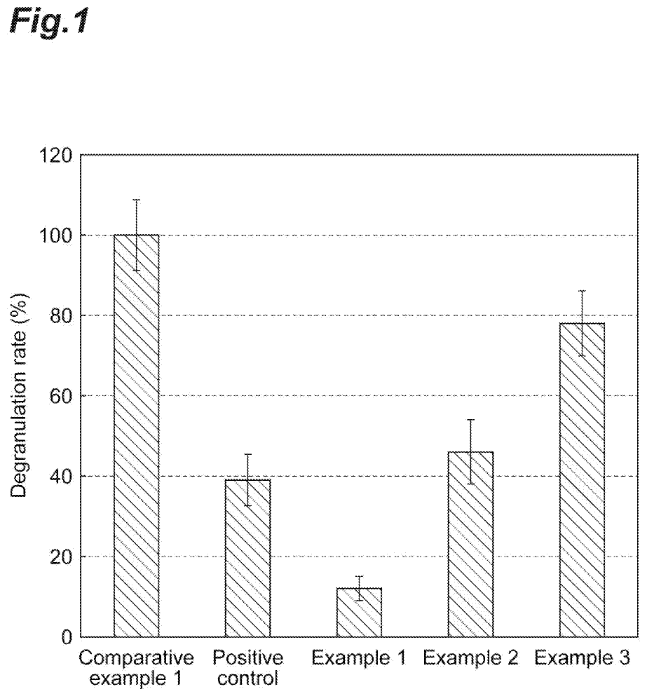

Results of calculating the degranulation rates are shown in FIG. 1. The degranulation rates of the positive control and Examples 1 to 3 were respectively 39±6.4, 12±3.0, 46±8.0, and 78±8.1, whereas the degranulation rate of Comparative Example 1 was 100±8.8.

<Test 2: Evaluation as Anti-Dementia Agent>

In the following description relating to the dose of p-coumaric acid, “100 mg/kg” means that 100 mg per kilogram of body weight was administered, for example. In some cases, “amyloid β proteins” may be simply referred to as “amyloid β.”

[Preparation of Test Solution]

p-Coumaric acid (trans-p-coumaric acid, Tokyo Chemical Industry Co., Ltd.) was stored at room temperature (control temperature: 18.0° C. to 28.0° C.) until it was used in a test. Injection water (Otsuka distilled water, Otsuka Pharmaceutical Factory, Inc.) was prepared as a medium in which p-coumaric acid was to be dissolved. The required amount of p-coumaric acid was weighed out and dissolved in the injection water. Then, the dissolved p-coumaric acid was diluted to a predetermined concentration to obtain a test solution.

[Preparation of Amyloid (3 Solution]

Amyloid β (Amyloid-β Protein (25-35, Polypeptide Laboratories) to be used in an amyloid β solution was stored in a freezer (control temperature: −30° C. to −20° C.) until it was to be used in a test. Amyloid β was dissolved in injection water to 2 mM to prepare an amyloid β solution.

[Test Animals]

Male Slc:ddY mice (SPF, Japan SLC, Inc.) were used as test animals 5-week-old mice were obtained as the mice. Mice are animal species generally used in behavioral pharmacology tests, and maintenance of their strains is clear. The body weight range of the mice 1 day after the acquisition was 23.8 to 30.0 g. The obtained mice had a preliminary breeding period of 5 days.

(Breeding Conditions)

The mice were raised in an animal breeding room which was maintained to have a control temperature of 20.0° C. to 26.0° C., a control humidity of 40.0% to 70.0%, 12 hours each of light and dark (lighting: 6 am to 6 pm), and a ventilation frequency of 12 times/hour (with fresh air passed through a filter).

Plastic cages (W: 310×D: 360×H: 175 mm) were used for group breeding up to 10 mice per cage from the preliminary breeding period to the day of grouping and for group breeding up to 5 mice per cage after the grouping. Solid feed (MF, Oriental Yeast Co., Ltd.) and tap water were freely fed as feed and drinking water, respectively.

[Test Operation]

(Group Configuration and Amount of Solution Administered)

Grouping was performed through a random sampling method on the start day of administration of a test solution so that the average body weight of each group was approximately uniform. As a group configuration, there were three groups: a pseudo-operation group, a medium control group, and a p-coumaric acid administration group. In the p-coumaric acid administration group, the amount of solution administered was calculated as 10 mL/kg based on the body weight on the administration day so that the amount of p-coumaric acid administered was 100 mg/kg per mouse. A 0.5% (w/v) methyl cellulose solution was administered to the pseudo-operation group and the medium control group at 10 mL/kg.

(Experimental Schedule)

The start day of administration of a test solution was regarded as day 1 of administration, the test solution was administered once a day, and an amyloid β solution was injected into the mice on day 8 of the administration. Thereafter, a Y-shaped maze test was carried out on day 14 of the administration. Each procedure will be described below.

(Administration Route and Administration Method of Test Solution)

The administration route was oral administration. Regarding the administration method, a test solution was orally administered according to a usual method used in test facilities using a polypropylene disposable injection cylinder (TERUMO CORPORATION) equipped with a disposable oral sonde for mice (Fuchigami Kikai). At the time of the administration operation, the test solution was mixed by inversion every time it was administered to a mouse, and was then sucked into the injection cylinder. On the day of injection of an amyloid β solution, the test solution was administered after the injection of the amyloid β solution. On the Y-shaped maze test day, the test solution was administered 30 minutes before measurement.

(Method for Injecting Amyloid (3)

The mice were anesthetized by intraperitoneally administering (amount of solution administered: 10 mL/kg) pentobarbital sodium (Tokyo Chemical Industry Co., Ltd.) to the mice at 40 mg/kg. After the anesthesia, levobupivacaine hydrochloride (Popscaine (registered trademark) 0.25% injection, Maruishi Pharmaceutical Co., Ltd.) was subcutaneously administered (0.1 mL) to the scalp. The scalp was incised to expose the skull, and a hole for stainless steel pipe insertion was made in the skull 1 mm lateral to (on the right side) and 0.2 mm posterior to the bregma using a dental drill. A stainless steel pipe which had an outer diameter of 0.5 mm and was connected to a silicon tube and a microsyringe was vertically inserted to a depth of 2.5 mm from the bone surface. For the p-coumaric acid administration group and the medium control group, 3 μL (6 nmol/3 μL) of an amyloid β solution was injected into the ventricles of the brain over 3 minutes with a microsyringe pump. On the other hand, 3 μL of injection water was injected into the pseudo-operation group through the same method. After the injection, the stainless steel pipe was allowed to stand for 3 minutes while being inserted into the hole, and was slowly removed. Thereafter, the skull hole was closed with a non-absorbable bone marrow hemostatic agent (Nestop (registered trademark), Alfresa Pharma Corporation), and the scalp was sutured.

(Evaluation Through Y-Shaped Maze Test)

A Y-shaped maze test (for example, Non Patent Literature 1) known as a method for evaluating learning and memorizing behaviors, particularly as a method for evaluating short-term memory was carried out. A plastic Y-shaped maze (Unicorn) which had three arms branching at 120 degrees to each other and in which one arm had a length of 39.5 cm, the floor had a width of 4.5 cm, and the wall had a height of 12 cm was used in the test.

Before evaluation, the illuminance at the floor surface of the device was controlled such that it was 10 to 40 lux. The evaluation was carried out 30 minutes after administration of a test solution. Mice were placed in either arm of the Y-shaped maze and allowed to freely explore within the maze for 8 minutes. The order of arms to which the mice moved within the measurement time was recorded, and the number of times the mice moved between the arms was counted to obtain a total entry number. Next, among these, combinations in which three different arms were continuously selected were examined to obtain a spontaneous alternation behavior number. The spontaneous alternation behavior rate was calculated using the following equation.

Spontaneous alternation behavior rate (%)=[Spontaneous alternation behavior number/(total entry number−2)]×100

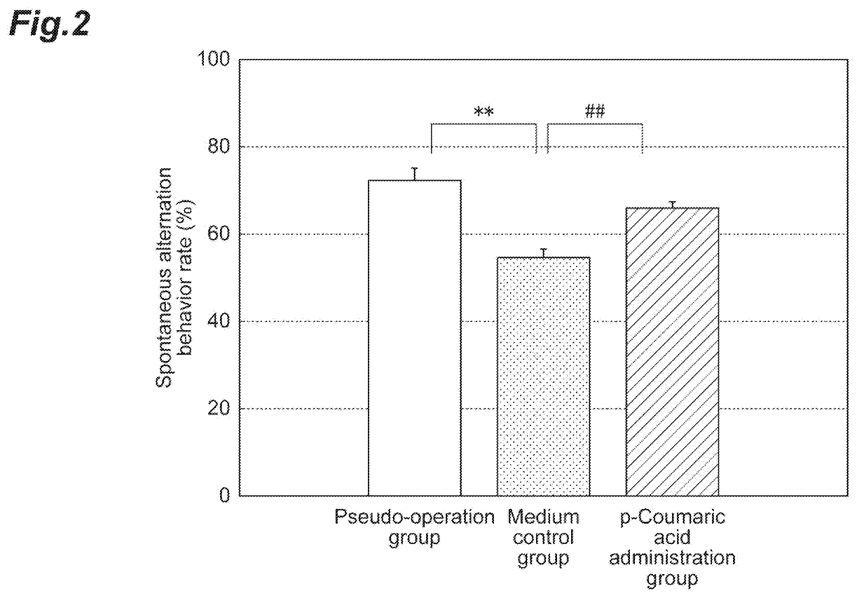

The Y-shaped maze test was performed on the mice of each group, and an average value and a standard error of the total entry number, the spontaneous alternation behavior number, and the spontaneous alternation behavior rate were calculated. Significant difference tests were compared between two groups between the pseudo-operation group and the medium control group and between the medium control group and the p-coumaric acid administration group. A homoscedasticity test through an F-test was performed for the comparison tests between two groups. A Student's t-test was performed in a case of equal variances, and an Aspin-Welch test was performed in a case of unequal variances. The significance level was set to significance percentage of 1%. A commercially available statistical program (SAS system, SAS Institute Japan) was used for the significant difference test. The results are shown in Table 2 and FIG. 2. As shown in Table 2 and FIG. 2, the spontaneous alternation behavior rate of the medium control group was lower than that of the pseudo-operation group, whereby a significant difference therebetween was recognized (p<0.01). In addition, the spontaneous alternation behavior rate of the p-coumaric acid administration group was higher than that of the medium control group, whereby a significant difference was recognized (p<0.01).

TABLE 2

| | | | p-Coumaric |
|---|---|---|---|
| | | | acid |
| | Pseudo-operation | Medium control | administration |
| | group | group | group |
| Total entry | 34.2 ± 1.6 | 36.4 ± 2.0 | 34.2 ± 2.1 |
| number | | | |
| Spontaneous | 23.1 ± 1.1 | 18.6 ± 1.0 | 21.3 ± 1.5 |
| alternation | | | |
| behavior | | | |
| number | | | |
| Spontaneous | 72.3 ± 2.8 | 54.6 ± 2.0 | 66.0 ± 1.4 |
| behavior | | | |
| alternation rate | | | |
| (%) | | | |

1-5. (canceled)

6. A method for inhibiting type I allergies comprising:
administering an effective amount of an agent comprising p-coumaric acid to a subject in need thereof.

7. The method according to claim 6, wherein the agent is based on a degranulation inhibitory action of mast cells or basophils.

8. The method according to claim 6, wherein the agent consists of p-coumaric acid.

9. The method according to claim 6, wherein the agent further comprises at least one material selected from the group consisting of amino acids, proteins, carbohydrates, oils and fats, sweeteners, minerals, vitamins, flavorings, excipients, binders, lubricants, disintegrators, emulsifiers, surfactants, bases, solubilizers and suspending agents.

10. The method according to claim 6, wherein the subject is human.

11. The method according to claim 6, wherein the agent is administered orally.

12. The method according to claim 6, wherein the agent comprises the p-coumaric acid in an amount greater than or equal to 100 μg/g and less than or equal to 700 μg/g based on the total amount of the agent.

13. The method according to claim 6, wherein the p-coumaric acid is administered in an amount greater than or equal to 40 μg per administration.

14. The method according to claim 6, wherein the p-coumaric acid is administered in an amount greater than or equal to 120 μg and less than or equal to 80 mg per day

15. A method for inhibiting degranulation of basophils and mast cells comprising:
administering an effective amount of a degranulation inhibitor for basophils and mast cells comprising p-coumaric acid to a subject in need thereof.

16. A method for improving/inhibiting dementia or short-term memory impairment comprising:
administering an effective amount of an agent comprising p-coumaric acid as to a subject in need thereof.
